(19) United States
(12) Patent Application Publication
(10) Pub. No.: US 2023/0233721 A1
(43) Pub. Date: Jul. 27, 2023

(54) SANITATION UNIT

(71) Applicant: Quanta Associates, L.P., Houston, TX (US)

(72) Inventors: Tanner Barbee, Houston, TX (US); Pat Latona, Galveston, TX (US); Robert Cantu, Pearland, TX (US)

(21) Appl. No.: 18/159,532

(22) Filed: Jan. 25, 2023

Related U.S. Application Data
(60) Provisional application No. 63/302,859, filed on Jan. 25, 2022.

(51) Int. Cl. A61L 2/20; A61L 2/26; A61L 2/24

(52) U.S. Cl. CPC A61L 2/208; A61L 2/26; A61L 2/24; A61L 2202/11; A61L 2202/122; A61L 2202/14; A61L 2202/15

(57) ABSTRACT

The disclosure relates to a sanitation and sanitization apparatus and methods for disinfecting a wearable equipment used in the catalyst reactor field including: a case having a first chamber and a second chamber; an internal assembly located within the first chamber of the case; a condenser within the internal assembly; an atomizer assembly within the internal assembly and connected to the condenser; a circulating pump within the internal assembly; an programmable logic controller configured to communicate with the condenser, atomizer, and circulating pump; and wherein the internal assembly has a housing containing the condenser, the atomizer assembly, the circulating pump, and the programmable logic controller; and a disinfectant port is defined on a side of the housing adjacent to the second chamber of the case.

### STATEMENTS REGARDING FEDERALLY SPONSORED RESEARCH OR DEVELOPMENT

Not Applicable.

### NAMES OF THE PARTIES TO A JOINT RESEARCH AGREEMENT

Not Applicable.

### BACKGROUND

### Technical Field

The disclosure relates to the portable and rapid sanitation and sanitization of wearable equipment, especially shared equipment while decreasing worker exposure to harmful chemical compounds.

Since the onset of the Coronavirus (SARS-COV-2) Pandemic, the ability to disinfect frequently used and shared items in a workplace has become central to maintaining healthy working environments. The current spread of the novel Coronavirus across the globe has highlighted concerns about practices surrounding the disinfecting of equipment that is used by multiple people, increasing the risk of exposure to viral and bacterial pathogens. There are many methods that currently provide the necessary disinfection level that are required; however, these rely on heavy oxidizers such as Ozone, Ethylene Oxide, and Quaternary Ammonium to kill bacteria and viruses on the surfaces. These processes require large amounts of time while also requiring precise control over the pressure and temperature present. Additionally, the chemicals react strongly with many of the materials that are present in the currently available helmets (or other industrial respiratory protection devices or wearable equipment), resulting in premature degradation of seals, other plastics, and possibly the failure of the fiberglass shell and rendering this equipment unfit or dangerous for use after only one or only a few disinfection cycles.

Catalyst removal, change-out or catalyst reactor maintenance requirements present a challenging work environment within an inert confined space in which respirator systems are required. The respirator systems include helmets. Helmets in the catalyst reactor field are often used repeatedly for days on end. Workers in this field often need to operate in a confined space and require respirators, including helmets. The work equipment, including uniforms, suits, and helmets, is often shared and thus need to be disinfected between uses. The individual parts of the equipment are often difficult to manually clean or disinfect by hand. The equipment, including the helmet, requires cleaning between uses and disinfecting. The recent epidemic of COVID-19 spreading globally has caused an increase in attention to proper disinfection and sanitation practices. Currently there are no products/processes on the market that provide high-level disinfection to wearable products such as helmets/respirators without partially or completely disassembling the unit, which requires valuable time and expertise to both disassemble and subsequently reassemble.

Conventionally, respirators are to be disassembled by removing speaking diaphragms, demand and pressure-demand valve assemblies, hoses, or any components recommended by manufacturer. Academy, T., Academy, R., & Trakt, S. S., Respirator Cleaning Procedures (Mandatory), United States Department of Labor—Occupational Safety and Health Administration (1998). All components must then be washed with mild detergent and rinsed completely. Soaking in a hypochlorite or aqueous iodine solution is also recommended when the initial cleaner does not contain a disinfectant. Id. These disinfectants often leave behind residual chemicals which can act as irritants to the user and will require thorough rinsing and wiping down after disinfection. The thorough cleaning of respirators requires a large amount of disassembly and exposure to large volumes of cleaning agents. This also poses a problem for a helmet system that additionally houses electronics. To prevent damage to delicate components and reduce turn-around between cleanings, a method that follows these guidelines without the explicit need for complete disassembly of the respirator would be preferable.

Most conventionally available disinfection methods rely on chlorine, ammonia, oxidizers such as ozone or hydrogen peroxide in a solution or UV-C light to interact with the organic compounds that make a bacteria's cell wall and virus's protein outer layer or in the case of UV-C light disrupt the production of RNA. Although sterilization would be ideal for all surfaces that will be in contact with the end user, the sterilization process is harsh and time consuming.

Companies such as STERIS, currently in the market of healthcare sanitation produce equipment that utilizes Ethylene Oxide Sterilization and Hydrogen Peroxide Vapor or Plasma as effective methods of sterilization, but require the tools undergoing sterilization to undergo temperature, humidity, and pressure fluctuations throughout the process. STERIS. Anatomy of an Ethylene Oxide Sterilization Process, available at https://www.steris-ast.com/wp-content/uploads/2016/03/10-Anatomy-of-an-Ethylene-Oxide-Sterilization-Process.pdf, last accessed on Nov. 18, 2021 (2020); Watling, D., Ryle, C., Parks, M., & Christopher, M., Theoretical analysis of the condensation of hydrogen peroxide gas and water vapour as used in surface decontamination, PDA Journal of Pharmaceutical Science and Technology, 56(6), pages 291-299, (2002). The duration required, approximately 1 to 6 hours, makes these prohibitively time consuming for use in the field. Finally, obtaining machines capable of sterilizing the volume required by the helmets range from $5,000-$20,000 based on a short search for machines available for consumer purchase, which can also be prohibitively costly.

Commercially available cleaning products require manual dilution and application of the disinfectant to surfaces. This exposes workers to chemicals that can pose potential health risks if not handled appropriately. Additionally, the improper removal of residue of certain disinfectants from the surface of equipment that will come into contact with skin or sensitive membranes such as the eyes or mouth can result in irritation of the contact area or chemical burning. United State Environmental Protection Agency, Reregistration eligibility decision for aliphatic alkyl quaternaries (DDAC), United State Environmental Protection Agency, August, pages 1-115, available at https://archive.epa.gov/pesticides/reregistration/web/pdf/ddac_red.pdf, last accessed on Nov. 18, 2021 (2006).

There have also been manufacturers of at home continuous positive airway pressure (CPAP) machines marketing products that claim to sanitize the breathing assistant machines. While the use of ozone has been widely used as a disinfectant for water there is little evidence that it is an effective agent in sterilizing facilities in general (i.e., hospital rooms). Environmental Protection Agency, U. S., Wastewater Technology Fact Sheet Ozone Disinfection, United States Environmental Protection Agency, pages 1-7, (1999). The Environmental Protection Agency (EPA) released a warning in February of 2020 stating that it had not cleared the use of ozone producing machines for the disinfection of at home devices. United State Environmental Protection Agency, Ozone Generators that are Sold as Air Cleaners, available at https://www.epa.gov/indoor-air-quality-iaq/ozone-generators-are-sold-air-cleaners#main-content and last accessed on Nov. 18, 2021 (2020). Moreover, ozone production is difficult to control and the product itself is hazardous to humans when exposed to the nose and eyes. (NIOSH), T. N. I. for A. S. and H., Ozone, available at https://www.cdc.gov/niosh/npg/npgd0476.html and last accessed on Nov. 18, 2021 (2019). The inability to properly control ozone production and the problem of disposal of the waste gas in a confined environment, along with the corrosion of critical components eliminates ozone disinfection methods as a plausible solution to the decontamination of helmets.

Accordingly, a need exists for a device with the capability to disinfect the frequently used commander helmets and other wearable equipment while in use in the field without exposing workers to harmful chemical compounds. The proposed exemplary embodiments aim to fully disinfect all surfaces of helmets or other wearable equipment, while preserving the functionality and durability of the equipment.

### SUMMARY

The disclosure relates to a sanitation and sanitization apparatus and methods for disinfecting a wearable equipment used in the catalyst reactor field including: a case having a first chamber and a second chamber; an internal assembly located within the first chamber of the case; a condenser within the internal assembly; an atomizer assembly within the internal assembly and connected to the condenser; a circulating pump within the internal assembly; an programmable logic controller configured to communicate with the condenser, atomizer, and circulating pump; and wherein the internal assembly has a housing containing the condenser, the atomizer assembly, the circulating pump, and the programmable logic controller; and a disinfectant port is defined on a side of the housing adjacent to the second chamber of the case.

### BRIEF DESCRIPTION OF THE DRAWINGS

The exemplary embodiments may be better understood, and numerous objects, features, and advantages made apparent to those skilled in the art by referencing the accompanying drawings. These drawings are used to illustrate only exemplary embodiments, and are not to be considered limiting of its scope, for the disclosure may admit to other equally effective exemplary embodiments. The figures are not necessarily to scale and certain features and certain views of the figures may be shown exaggerated in scale or in schematic in the interest of clarity and conciseness.

FIG. 1 depicts an exemplary embodiment of a disinfection or sanitation device or unit.

FIG. 2 depicts an isometric view of the internal assembly of the exemplary embodiment of the disinfection or sanitation device or unit.

FIG. 3 depicts a top view of the internal assembly of the exemplary embodiment of the disinfection or sanitation device or unit.

FIG. 4 depicts a side view of the internal assembly of the exemplary embodiment of the disinfection or sanitation device or unit.

FIG. 5 depicts an end view of the internal assembly of the exemplary embodiment of the disinfection or sanitation device or unit.

FIG. 6 depicts an exploded isometric view of the condenser or humidifier of the exemplary embodiment of the disinfection or sanitation device or unit.

FIG. 7 depicts a side cross-section view of the condenser or humidifier of the exemplary embodiment of the disinfection or sanitation device or unit.

FIG. 8 depicts an end view of the condenser or humidifier of the exemplary embodiment of the disinfection or sanitation device or unit.

FIG. 9 depicts an exploded and enlarged isometric view of the condenser or humidifier of the exemplary embodiment of the disinfection or sanitation device or unit.

FIG. 10 depicts an exploded isometric view of the atomizer of the exemplary embodiment of the disinfection or sanitation device or unit.

FIG. 11 depicts an end view of the container or case for the exemplary embodiment of the disinfection or sanitation device or unit.

FIG. 12 depicts a top view of the exemplary embodiment of the disinfection or sanitation device or unit, without the helmet or disinfection target within the case or container.

FIG. 13 depicts an enlarged view of the exhalation valve plug and the refill port of the exemplary embodiment of the disinfection or sanitation device or unit.

FIG. 14 depicts an enlarged view of the exhalation valve cradle of the exemplary embodiment of the disinfection or sanitation device or unit.

FIG. 15 depicts an enlarged view of the tubing and sensing port of the exemplary embodiment of the disinfection or sanitation device or unit and helmet.

FIG. 16 depicts a schematic diagram of the exemplary embodiment of the disinfection or sanitation device or unit and an exemplary embodiment of the airflow within the disinfection or sanitation device or unit.

FIG. 17 depicts an isometric view of a schematic diagram of an alternative exemplary embodiment of a disinfection or sanitation device or unit.

FIG. 18 depicts an isometric view of an alternative exemplary embodiment of a disinfection or sanitation device or unit with a helmet.

### DESCRIPTION OF EMBODIMENT(S)

The description that follows includes exemplary apparatus, methods, techniques, and instruction sequences that embody techniques of the inventive subject matter. However, it is understood that the described embodiments may be practiced without these specific details.

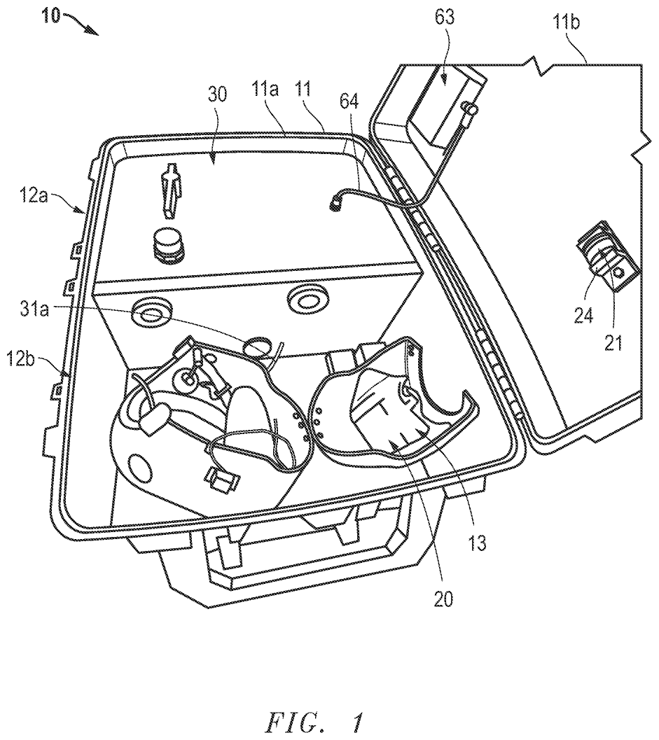

FIG. 1 depicts an exemplary embodiment of a disinfection or sanitization system, device or unit 10 for disinfecting or sanitizing a helmet or commander helmet 20 or other wearable equipment 13 in a timed cycle. The device 10 is portable, easily stored, and simple to use with little instruction. The device 10 cleans or disinfects as much of the surface of the helmet 20 as possible including: face seals, helmet surface, the rear bladder, mesh lining, and interior cavities where exhaled breath of the user is exhausted without causing premature wear to the components comprising the helmet 20, and disinfecting such areas that may not be accessible by conventional cleaning methods. The helmets 20 and other wearable equipment 13 to be disinfected may incorporate a variety of materials, including: stainless steel, fiber glass, aluminum, epoxy resin and hardener, silicone, acetal, PVC vinyl, nylon, neoprene, brass, and more.

The device 10 and the helmet 20 to be disinfected are enclosed in a box, case, casing or container 11. In certain exemplary embodiments, the container or casing 11 may be a 1630 Protector Transport Case as commercially available from PELICAN™, having dimensions of approximately 24 inches by 36 inches by 15 inches (or 60.96 cm by 91.44 cm by 38.1 cm), although other types and sizes of boxes, containers, and casings 11 may be used as is known to one of ordinary skill in the art. The device casing or container 11 may include a base 11a, and a lid or top 11b that is openable and securely closeable against the base 11a and prevents the unintended escape of air or liquids from the container 11. The base 11a may be generally partitioned or divided into a first chamber 12a and a second chamber 12b, wherein each chamber 12a, 12b takes up approximately half the space or area provided by base 11a. The internal assembly 30 may occupy the first chamber 12a; in exemplary embodiments, the first chamber 12a may be on the left side of the base 11a. The second chamber 12b may house the helmet 20 or other target disinfection item/wearable equipment 13 towards the right side of the base 11a in certain exemplary embodiments, or is otherwise configured for mounting of the helmet 20 or other item/wearable equipment 13. The lid 11b contains user interface components 63 which are connected to the internal assembly 30 (and electronics module 60) via wiring, cables, or data communication techniques 64, which may be wireless data communication in certain exemplary embodiments. The device casing or container 11 allows the device 10 to be moved and stored simply, while also providing the necessary enclosure to prevent accidental exposure to the disinfectant while the sanitation system or device 10 is in use. The primary purpose of the device 10 is for disinfection only. The device 10 is not currently intended to remove any dirt, sweat, grime, grease or any other material contaminate from the object/wearable equipment 13 or helmet 20 placed inside the container 11.

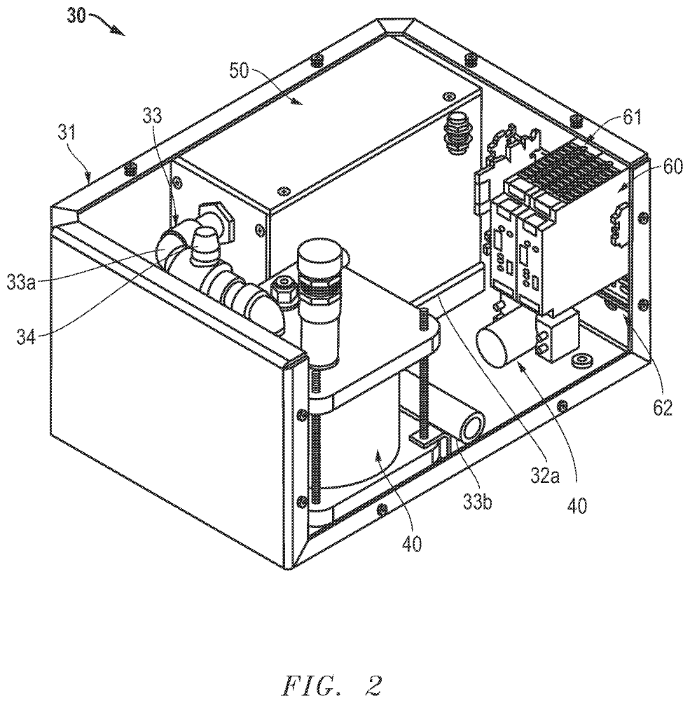

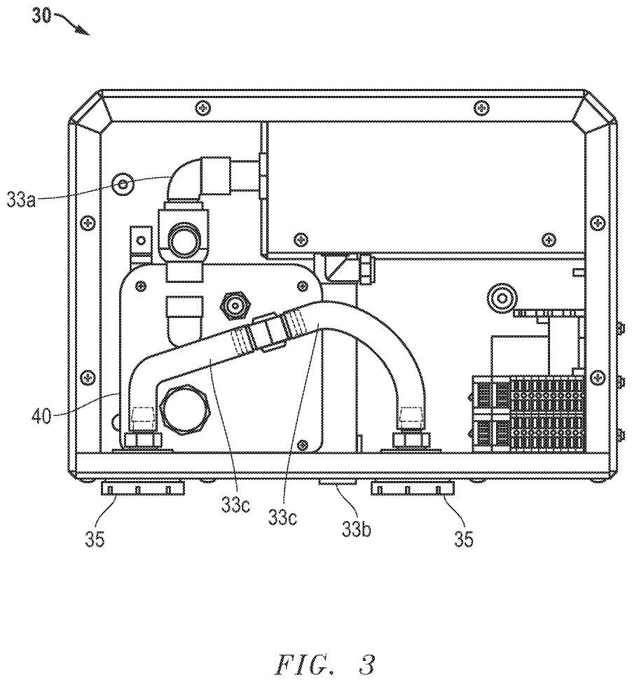

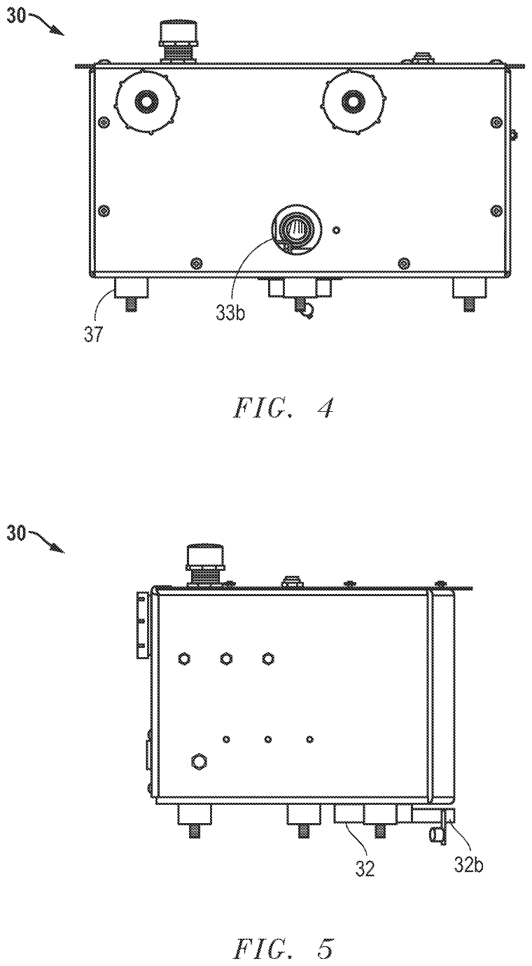

As is further depicted in FIGS. 1-5, the atomizer 40, dehumidifier or condenser 50, circulating pump 70, and electronic controls 60 are together part of the internal assembly or subassembly 30 housed in a stainless-steel container or housing 31 as located or secured inside a first chamber 12a of the device casing 11. The internal assembly container or housing 31 isolates delicate components such as the programmable logic controller (PLC) as part of the electronic controls 60, relays, and power supply units from the corrosive disinfectants or cleaning agents that will be used, and thus prolonging overall lifecycle of the device 10. The internal assembly 30 in the container 31 takes up approximately half of the internal space of the device casing 11, as a first chamber 12a of the device casing 11, and is bolted using vibration dampers 37 to the bottom 11a of the case 11. A drain pan 32 is located at the bottom rear of the housing 31 beneath the condenser 50, and collects moisture removed from the air by the dehumidifier or condenser 50. The drain pan 32 may also have a sloped edge 32a at a side to allow or enable the ease of collection of and to retain condensation from the dehumidifier 50. The drain pan 32 may also include a drain line 32b leading out of the case 11, which may be capped and uncapped for easy disposal of the condensation once use of the device 10 is completed. Stainless steel is an exemplary embodiment for the material of the assembly housing 31 due to its resistance to corrosion, although other materials as known to one of ordinary skill in the art may be selected, by way of example, molded materials including metal and plastics. While the weight penalty of stainless steel is apparent when lifting the device 10, lighter materials such as, by way of example, aluminum, are unable to withstand prolonged exposure to hydrogen peroxide or other disinfectants 80 as needed to sanitize the helmet 20 or other equipment 13 properly.

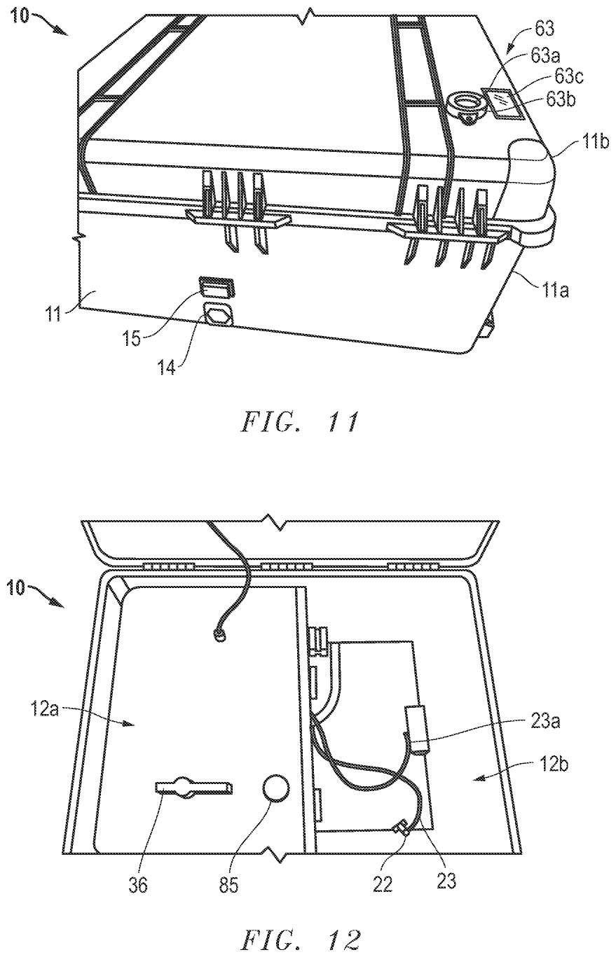

One or more tubes, pipes, ducts or lines 33 connects the atomizer 40, condenser 50, and disinfection target chamber 12b. The tube 33a connects the condenser 50 to the atomizer 40, which provides drier air to the atomizer 40. A ball valve 34 (and as controlled by flow control valve or valve handle 36, see FIG. 12) is located on the line 33a, so as to prevent contact of the disinfectant with the condenser 50 when the device 10 is not in use. The tube 33b is the return line from the sanitization chamber 12b to the condenser 50, wherein the humid or moist air is drawn from the chamber 12b to the condenser 50. There are at least two disinfectant ports 35 as defined on the housing 31 side that faces, abuts or is adjacent to the sanitization chamber 12b, and enables the fogged, misted or atomized disinfectant to travel from the atomizer 40 to the sanitization chamber 12b via lines 33c.

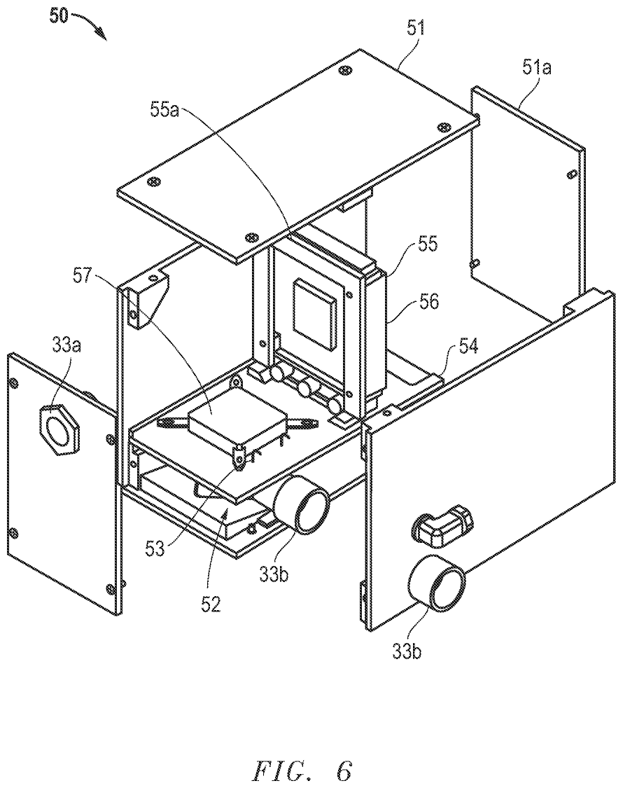

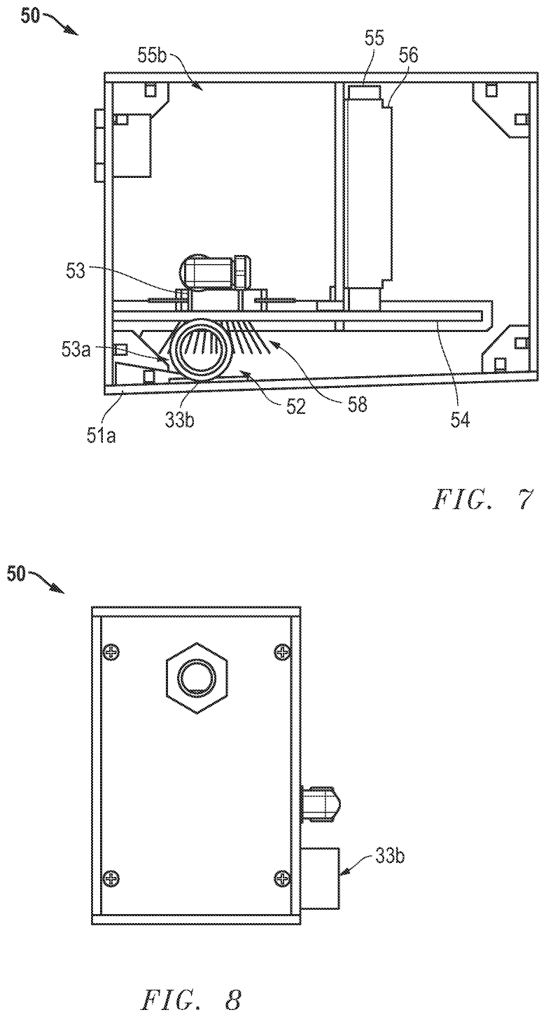

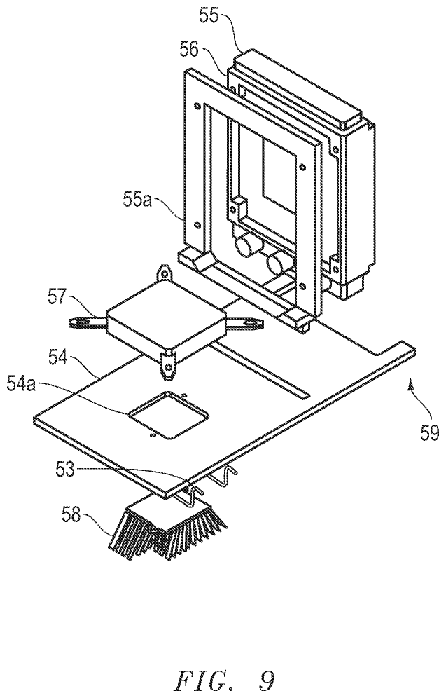

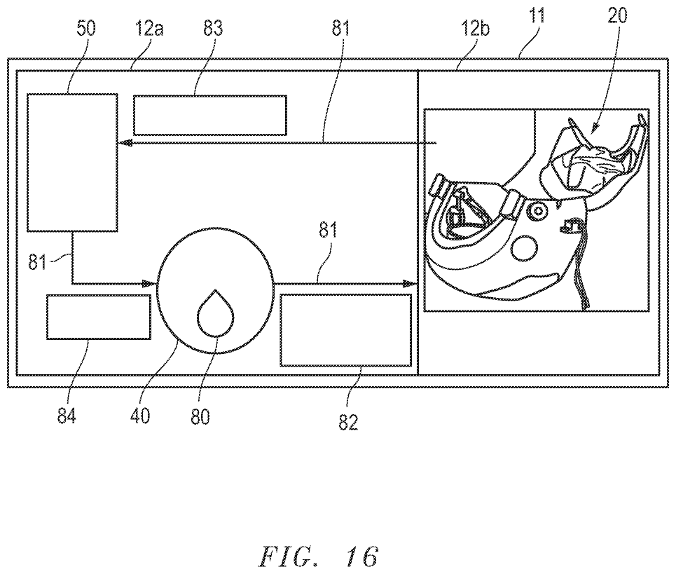

As depicted in FIGS. 6-9, the dehumidifier 50 acts to both remove moisture from the air and to circulate the air throughout the device 10 (see e.g., FIG. 16). The dehumidification assembly 50 is, in an exemplary embodiment, made of several pieces of polyvinyl chloride (PVC) plastic machined to construct the walls or borders 51a of the casing or body 51, and a supporting horizontal inner or internal shelf 59. A Peltier cooling device, or thermoelectric cooler 53 is secured to a cooler mount 54 on the internal shelf 59 and is utilized to create a cold zone 53a where the air flow enters. The cooler mount 54 may define a cooler mount opening or internal shelf opening 54a. The cold area 53a causes condensation to form on the heatsink 58 which collects in the drainage basin 52 beneath the inner shelf 54 and the heat sink 58. Condensation may also accumulate on the fan 55. The first or water-cooled heat sink 57 is secured above the cooler mount 54. The second heat sink 58 is secured below the cooler mount 54. The top of the Peltier thermal cooler 53 is connected to the water-cooled heat sink 57. This allows the Peltier thermal cooler 53 to operate at temperatures cold enough to condense water from the air. The drainage basin 52 which collects the water condensation may have a slope or angle to enable to water to flow and collect accordingly; further the bottom wall 51a of the body 51 may also be sloped or angled in the opposite direction of the drainage basin slope; although in alternative exemplary embodiments, the angle of the drainage basin 52 and the bottom wall 51a may slope in similar directions. The drainage basin 52 may also connect to the drain pan 32 of the housing 31 in case of overflow. The fan 55 that powers the overall sanitization system 10 also acts to cool the Peltier cooling device 53. The fan 55 and radiator 56 may both be mounted onto the fan mount 55a, which may vertically extend from and connect to the horizontal inner shelf 59 towards an end of the shelf 59, next to the heat sinks 57,58 and the cooling device 53. The fan 55 and radiator 56, in addition to the horizontal internal shelf 59 and the walls 51a of the casing 51, may create a fan chamber or enclosure 55b which provides the dehumidified air to the atomizer 40. The arrangement of the dehumidifier or condenser 50 results in warm, dehumidified air being pushed to the atomizing chamber 40 where the device 10 can more efficiently move the atomized hydrogen peroxide than if no liquid were removed or condensed from the air. Additionally, when the atomizer 40 stops producing fog the dehumidifier 50 acts to remove remaining atomized hydrogen peroxide from the helmet chamber 12b before the user opens the container 11, limiting any potentially hazardous exposure.

The disinfection or sanitation device 10 has, in an exemplary embodiment, been effectively measured at a 20% humidity drop across the dehumidifier unit 50. However, should the air become sufficiently hot the Peltier cooling device 53 may not be capable of maintaining the required temperature difference at a low enough “cold side” temperature and can possibly overheat, eliminating the chance of acceptable condensation forming. Further, the components of the dehumidifier unit 50 may be susceptible to corrosion when exposed to hydrogen peroxide for extended periods of time. By way of example, both heatsinks 57, 58, and the radiator 56 can be made from aluminum, and the fan 55 can potentially lose functionality if electronics of the fan 55 or the electronics module 60 become damp. Hence, components, usage times, and materials of construction may be adjusted as needed.

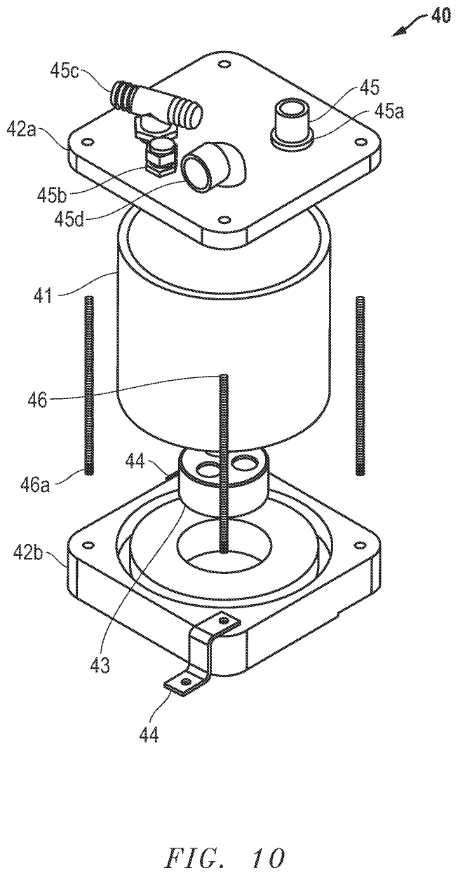

The atomizer assembly or atomizer 40, as depicted in FIG. 10, holds or contains the disinfectant that will be circulated throughout the system or device 10. In exemplary embodiments, the atomizer assembly 40 is constructed from PVC plastic, with a machined top casing or plate 42a and bottom casing, base, or plate 42b and a PVC plastic 6-inch (or 15.24 cm) pipe or tube that makes up the atomizer chamber or central body 41. Other materials as known in the art may be used to construct the parts of the atomizer assembly 40. A stainless-steel ultrasonic atomizer 43 is utilized to generate a fog or mist of the disinfectant liquid held in the atomizer chamber or container 41. The ultrasonic atomizer 43 is able to generate a fog or mist of the disinfectant liquid through harmonic oscillations or vibrations, and is powered by 24V DC in an exemplary embodiment. In certain exemplary embodiments, the atomizer 43 may be a vibrating plate. Physically dispersing the disinfecting agent, as opposed to heating into a vapor, is preferable as this limits the complexity of the overall device or system 10 by eliminating the need to monitor both pressure and temperature variations of the system 10 to determine the quality of the disinfecting agent 80 present. Fogging relies on the atomization of the disinfectant 80 as opposed to its vaporization. Fogging also has its limitations regarding what chemicals are viable for use.

The top and bottom casings 42a, 42b clamp to either side of the PVC pipe or atomizer chamber 41. Sealant is used to create a watertight barrier at the connecting faces between the casings or plates 42a, 42b and the atomizer chamber 41. Two or more fasteners or tie downs 44 hold or secure the atomizer 40 in place within the device container 11 and the internal assembly housing 31. Further, each corner of the top plate 42a, and the bottom plate 42b may have a hole defined for the insertion of a bolt 46 and insert nut 46a to secure the atomizer assembly 40 together. In the top plate 42a four (4) holes, ports, or openings 45 are cut or defined: a port 45a acting as a means for filling or refilling the chamber 41 with the disinfectant liquid; a port 45b to allow power into the ultrasonic atomizer 43 at the base of the chamber 41, a port 45c to allow fogged disinfectant air out to the disinfecting or sanitizing chamber 12b, and a port 45d to allow air into the atomizing chamber 41 from the condenser 50 as connected line 33a. A ball valve 34 is located on the line 33a connected to port 45d coming into the atomizer 40 to prevent spills from entering the condenser 50, and the fill tube connected to port 45a is capped, by way of example, with a screw cap 85.

The electronics controls, system, or module 60 may be best observed in in FIGS. 1-3 and 11. In an exemplary embodiment, the electronics system 60 is controlled by an Arduino-based PLC 61 as commercially available from INDUSTRIAL SHIELDS®, although alternative computing units or PLCs 61 may be used as known by one of ordinary skill in the art. The PLC 61 holds the program that times or schedules the activation of the atomizer 40, circulating pump 70, Peltier cooling device 53, and detects when the start/stop button 63a or other elements of the user interface 63 has been pressed. In the exemplary embodiments as depicted, the electronics controls 60 and all necessary relays and power supplies are mounted to DIN rail or other electronic rail 62 towards one side of the device 10 (as located within the internal assembly 30 stainless steel container 31). The electronics module 60 provides a simple means of programming, and controlling the system or device 10; further, more components can be added as needed in future development, and all voltages are a standard 24V DC that is common in industrial applications. The electronics module 60 may communicate to the components of the device 10 wirelessly or via cable or wire means 64.

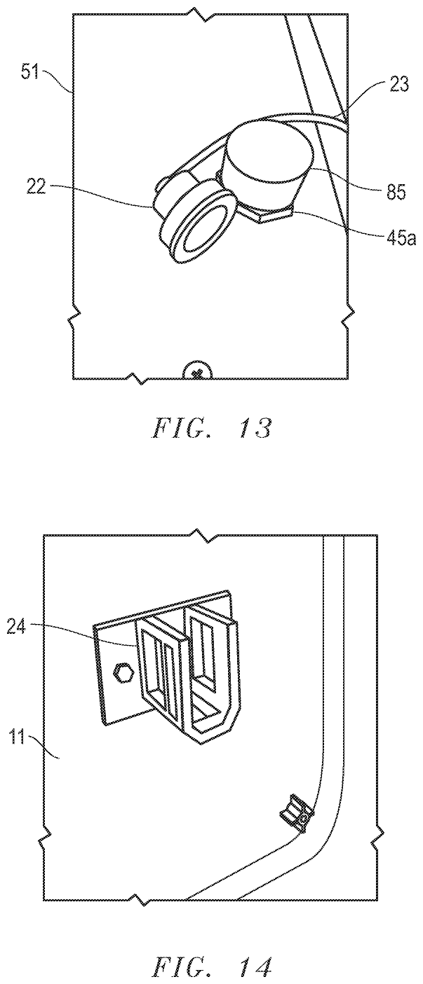

By way of example, as seen in FIG. 11, a light ring 63b, wherein the light ring 63b has or includes multiple LEDs arranged in a circle, can be used as an indicator for the internal status of the device 10 and provides at least three color indication statuses: a stand-by status (wherein the light ring may illuminate or flash yellow or orange); a ready status (wherein the light ring 63b may illuminate or flash green or white); and a warning status that hazardous materials are being circulated (wherein the light ring 63b may illuminate or flash red). The light ring 63b may, by way of example, be a NEOPIXEL light ring as available commercially from ADAFRUIT. In the depicted exemplary embodiment of FIG. 11, the start/stop button 63a may be located within or in the center of the light ring 63b. Further, an LCD or other display screen 63c may be integrated onto the lid 11b to display a more readable status and count-down timer and offer an additional or alternative method of user input. The user interface 63 may be protected or housed in a user interface housing 63d on the underside of the lid 11b. The circulating pump 70 located beneath the electronic or DIN rail 62 draws air from the inside of the helmet 20 through plugging the exhalation cut out on the inside bottom of the helmet 20 with the exhalation valve plug 22 (see e.g., FIG. 13) and connecting the sensing port 25 (see FIG. 15) at the top of the helmet 20 via tubing 23. The circulating pump 70 may also be connected to the helmet 20 exhalation valve plug 22 through the housing 31 opening(s) 31a and tubing 23. The outlet port of the circulating pump 70 is also connected to an input port of the condenser 50 via further tubing or piping 23. The plugged exhalation cut out and the sensing port 25 as connected through tubing 23 supplies the necessary suction to draw air through the internal cavities of the helmet 20 where the chances of the disinfectant passively entering are relatively small.

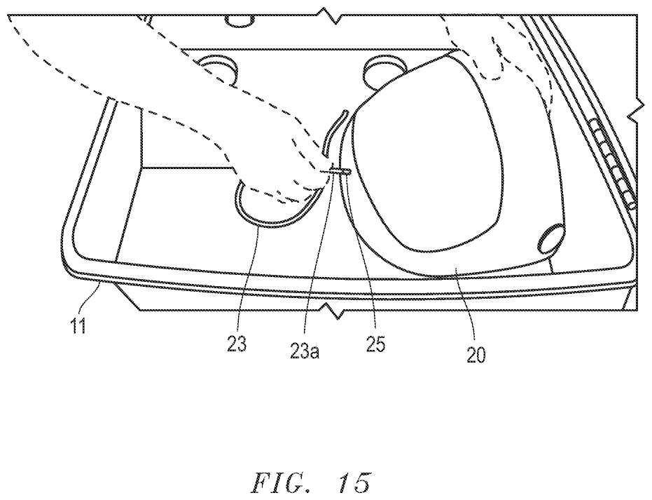

As best shown in FIG. 12, the flow valve control 36 must first be opened and turned or set to be in-line with the refill port 45a (this position of the flow valve control 36 is 90 degrees from the closed position), prior to operation, to allow the disinfection to begin. The operator should remove all pads, and any other material which may significantly deteriorate, from the interior of the commander helmet or helmet 20 or other targeted disinfection item 13. With a damp cloth, the user should then wipe down all surfaces to remove dirt, sweat, and any other contaminates from the helmet 20 or other targeted disinfection item 13. The exhalation valve 21 should be removed from the helmet 20 and placed into the exhalation valve cradle or holder 24 located on the inside of the lid 11b (see, e.g., FIGS. 1 and 14). The exhalation valve cradle or holder 24 has an open framework so as to allow the disinfectant fog to also sanitize the exhalation valve 21 when placed in the cradle 24. The user should check if there is liquid inside the atomizer chamber or container 41 via unscrewing the cap 85 at the fill port 45a to check and visually confirm accordingly (see e.g., FIG. 13). If needed, the user should remove the contents of the atomizer chamber or container 41 by: inserting a draw tube into the fill port 45a until it reaches the bottom of the atomizer container 41; then using a syringe to draw or withdraw any disinfectant 80 from the container 41 and dispose of the disinfectant 80, repeating as necessary until no more liquid is withdrawn. The user should then fill the container 41 with hydrogen peroxide disinfectant 80 by drawing the appropriate amount of hydrogen peroxide 80 from its storage bottle; placing tubing into the fill port hole 45a; depressing the syringe and repeating as necessary to completely fill the container 41. In certain exemplary embodiments, the user should fill the atomizer chamber or container 41 with an amount of approximately 250 mL of hydrogen peroxide disinfectant 80. The atomizer 40 may also be able to fog the disinfectant 80 with as little as 5 mL of the disinfectant 80.

The user should then connect the box power outlet 14 (as shown in FIG. 11) to power using a power cable. The power source in exemplary embodiments may be 110 V AC power, so as to allow users to be able to use the device 10 in a variety of environments without requiring a specialty power source. An on/off or power switch 15 may be flipped near the outlet 14 located on a side or back of the box 11 so that the indicator light ring 63b is illuminated. The light ring 63b on the top of the lid 11b should illuminate white or other color indicating a ‘ready status.’ The commander helmet 20 should be opened and placed into the chamber 12b inside the case 11 in a face-down position as shown in FIG. 1. The helmet 20 should have an opening or cut-out where the exhalation valve 21 was removed; the exhalation valve plug(s) 22 should be inserted into the exhalation valve opening or cut-out on the helmet. The exhalation valve plug(s) 22 is connected to a first end of the tubing 23; the other end of the tubing 23 is a tubing end 23a. The tubing end 23a should be connected or screwed into the helmet sensing port 25. The user should ensure that the helmet 20 is face-down in the chamber 12b, and then close and latch the lid 11b. The user should then depress the start/stop button 63a at the top of the lid 11b, which may be located at the front, upper left side of the lid 11b of the container 11. The indicator light ring 63b should light up or illuminate a yellow or other stand-by or cautionary color as the system 10 starts up. After a few minutes, the indicator light ring 63b may change to illuminate a single LED as red on the ring 63b, and the ring 63b may progressively illuminate or light up a greater portion of the ring 63b to be a red color, until the whole ring 63b may be illuminated red. During the time that any portion of the light ring 63b indicates, shows, or lights up a red or other warning or dangerous color, the case 11 should not be opened as the warning, dangerous or red color of the light ring 63b is indicative of the portion of the cycle where a dense fog 82 of the disinfectant 80 is present in the case 11.

FIG. 16 depicts an outline or schematic of the airflow 81 of the disinfection cycle within the disinfection or sanitation device or unit 10, during the cycle where one or more of the LEDs in the light ring 63b is illuminated a red or warning color. The airflow 81 as depicted in FIG. 16 and as described below allows for both the dispersal of disinfectant 80 into the target disinfection, helmet, or second chamber 12b and the removal of the atomized disinfectant 82 from the air within the helmet or second chamber 12b of the enclosure or casing 11. An example of a disinfectant 80 for the sanitation unit 10 is hydrogen peroxide at a concentration of at least 8%, and preferably an 8% pure food grade hydrogen peroxide. By way of further example, in certain alternative exemplary embodiments, the hydrogen peroxide may be further mixed with peracetic acid (0.85%) or silver (0.01%). The diluted hydrogen peroxide will quickly disinfect contacted areas while minimizing corrosion and damage to sensitive parts of the commander helmet 20 or other targeted disinfection target 13, including other kinds of wearable equipment. Use of other disinfecting agents 80 can result in poor performance of the device 10 and potential damage to the components 13, such as helmet 20, intended to be disinfected.

When the start/stop button 63a is pushed or engaged and the container 11 is closed, the disinfection unit, device or system 10 functions by creating a fine mist of droplets or a fog of disinfectant 82 through the implementation of an atomizer 40 in a first chamber 12a and propelling or exhausting an even dispersal of the atomized disinfectant 82 into the second chamber 12b holding the helmet 20. The concentration of hydrogen peroxide of the fog 82 will mirror the concentration of the liquid solution 80 that is fed into the system 10 (i.e., an 8% hydrogen peroxide liquid disinfectant 80 should atomize into an 8% hydrogen peroxide fog 82). The humid or moist air 83 (i.e., from the second chamber 12b) is then drawn into a dehumidifier where excess fog 82 is collected and drier air 84 (e.g., dried air 84 from condenser 50) is recycled to the atomizer 40. A fan 55 located in the dehumidifier or condenser 50 (which, by way of example, may be a 12V fan 55) provides the air flow 81 necessary to move the disinfectant fog 82, moist air 83, and drier air 84 within the device or system 10 as described. The byproduct of the reaction of hydrogen peroxide is simply water so therefore post treatment wipe-down, and rinsing is not necessary as there are not harmful residues expected to be left on the surfaces of the device 10 and helmet 20 (or other wearable equipment 13) once the process is complete; although in certain instances, a wipe-down step may be warranted or prudent as an extra precautionary measure (including instances when the box or container 11 is opened sooner than expected or desired). Further, it is vital to consider possible negative interactions between the disinfectant solution 80 and the surfaces of the device 10 and helmet 20 that the disinfectant 80 will contact. Temporary exposure to the active ingredients in the choice of disinfectant 80 may result in “none” to “inconsequential” wear on parts. However, the use of hydrogen peroxide has been shown to have minimal immediate deterioration to most of the vulnerable fabrics such as nylon, polyester, wool, and Nomex®, and does not react as strongly with the remainder of the range of materials that is generally present on the helmet 20 or other wearable equipment 13 (as compared with other disinfectants conventionally available).

At the end of the disinfection cycle, the ring 63b will light up a yellow or other cautionary or stand-by color again, and will hold this color for a few minutes while the device 10 clears out the remainder of the fog 82 and the atomizer or mist-maker 43 stops producing the fog 82. When the disinfection or sanitation cycle is complete, the light ring 63b will flash or illuminate a green and then illuminate a white light about the ring 63b; other combinations of green and white LED lights can be used on the user interface 63 to indicate a safe or ready status. Further, these status indications as described herein can be displayed to the user via other means, such as the display screen 63c. The helmet 20 or other wearable equipment 13 can now be removed from the case 11 and inspected for any residual condensation. The user may wipe down any remaining condensation from the helmet 20 or other wearable equipment 13. The equipment 13 or helmet 20 should be allowed to dry before storing or reusing.

The entirety of the disinfection cycle, between depressing the ‘start’ button 63a to start or engage the cycle and when the disinfection cycle finishes and indicates that the case 11 is ready to open, can be approximately 5 to 10 minutes in total. The time during when the fog 82 is circulating within the device 10 and the light ring 63b illuminates red, may only be 3 to 5 minutes in total, and the remainder of the time, perhaps 2 to 3 minutes, may be the device 10 conditioning (or starting up), and clearing out the fog 82. This time frame enables users to disinfect helmets 20 quickly and portably instead of having to spend valuable time disassembling and manually disinfecting each piece of the helmet 20, or spend hours disinfecting in other bulky and costly alternative conventional methods. This quick time frame also minimizes downtime in the field, especially with regards to catalyst reactor work.

In order to shut down the disinfection or sanitation unit 10, the power to the case 11 should be turned off via the switch 15 and the power disconnected or unplugged from the outlet 14. The user should ensure that the case 11 is empty (i.e., no helmet 20 or other equipment 13 remains in the case). The user should unscrew the cap 85 at the fill port 45a and check if there is liquid inside the atomizer chamber or container 41 (see e.g., FIG. 13). If there is any disinfectant 80 remaining, the user should remove the contents of the atomizer chamber or container 41 by: inserting a draw tube into the fill port 45a until it reaches the bottom of the atomizer container 41; then using a syringe to withdraw the disinfectant 80 from the container 41 and dispose of the disinfectant 80, repeating as necessary until no more liquid is withdrawn. Any remaining or residual liquid disinfectant 80 or condensation should be wiped dry from the device 10. The drain pans 32 and/or drainage basins 52 may also be emptied at this step by uncapping the drain line 32b and allowing any fluid or condensation to be dumped, relieved, or removed from the case 11. The lid 11b can be closed and the device 10 can now be stored, preferably with the lid 11b facing upwards. Before moving the case, the flow control valve 36 should be turned 90 degrees to the right in the closed position (i.e., the valve control handle 36 should not be in-line with the fill port 45a and screw cap 85).

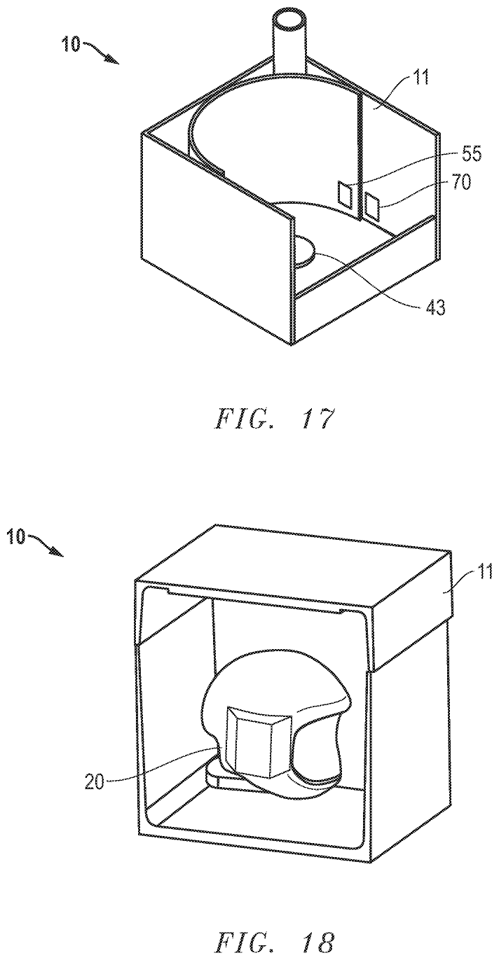

In a further alternative exemplary embodiment as depicted in FIGS. 17-18, a small box, container, casing 11 large enough to house a single helmet 20 for disinfection, approximately two cubic feet, is fitted with the appropriate components from internal assembly 30 to accomplish disinfection. A pool of liquid hydrogen peroxide or other disinfectant 80 is accumulated at the floor of the container 11, and an ultrasonic vaporizer or atomizer 43 is used to produce a fog of the pooled disinfectant 80 in a single chamber within the container or box 11. Once a fog forms, the disinfectant fog 80 can then be circulated throughout the container 11 by the use of one or more fans 55 directing outside air into the container 11 and drawn through difficult to access portions of the helmet 20 such as the manifold through the use of a small pump 70 attached to one or more ports at the front of the helmet 20 (note, optionally the fan 55 and pump 70 could be combined as a fan proximate ultrasonic vaporizer or atomizer 43). The device 10 will require a timer to ensure the surfaces receive enough exposure to the disinfectant fog 80, approximately 20 to 30 minutes.

While the embodiments are described with reference to various implementations and exploitations, it will be understood that these embodiments are illustrative and that the scope of the inventive subject matter is not limited to them. Many variations, modifications, additions, and improvements are possible.

Plural instances may be provided for components, operations or structures described herein as a single instance. In general, structures and functionality presented as separate components in the exemplary configurations may be implemented as a combined structure or component. Similarly, structures and functionality presented as a single component may be implemented as separate components. These and other variations, modifications, additions, and improvements may fall within the scope of the inventive subject matter.

1. A sanitation apparatus for disinfecting a wearable equipment used in the catalyst reactor field comprising:
a case having a first chamber and a second chamber;
an internal assembly located within the first chamber of the case;
a condenser within the internal assembly;
an atomizer assembly within the internal assembly and connected to the condenser;
a circulating pump within the internal assembly;
a programmable logic controller configured to communicate with the condenser, atomizer, and circulating pump; and
wherein the internal assembly comprises a housing containing the condenser, the atomizer assembly, the circulating pump, and the programmable logic controller, and wherein a disinfectant port is defined on a side of the housing adjacent to the second chamber of the case.

2. The apparatus of claim 1, wherein the atomizer assembly further comprises
an atomizer chamber having a top plate sealed to the top of the atomizer chamber, and a bottom plate sealed to the bottom of the atomizer chamber; and
an ultrasonic atomizer mounted on the bottom plate.

3. The apparatus of claim 2, wherein the top plate of the atomizer chamber defines: a first port to provide a fogged disinfectant to the disinfectant port; a second port connected to the condenser; and a refill port defined through the housing of the internal assembly.

4. The apparatus of claim 3, wherein the condenser further comprises
a condenser casing having an internal shelf within the casing, wherein the internal shelf is horizontal within the casing;
a drainage basin beneath the internal shelf;
a cooling device mounted on the internal shelf; and
a fan mounted vertically to the internal shelf.

5. The apparatus of claim 4, wherein the condenser further comprises
a first heat sink mounted above the cooling device, wherein the first heat sink is a water-cooled heat sink, and further wherein a top of the cooling device is connected to the first heat sink; and
a second heat sink mounted beneath the internal shelf and the cooling device, wherein the second heat sink is above the drainage basin.

6. The apparatus of claim 5, wherein the second port of the atomizer chamber is connected to the condenser via a first tube; and further comprising a ball valve located on the first tube, wherein the ball valve is controlled by a valve handle on the housing.

7. The apparatus of claim 6, further comprising a second tube connecting the second chamber of the case to the condenser casing.

8. The apparatus of claim 7, wherein the condenser casing, the fan, and the internal shelf form an enclosure configured to provide dehumidified air to the atomizer.

9. The apparatus of claim 8, further comprising a user interface in data communication with the programmable logic controller, wherein the user interface comprises a light ring having a plurality of LEDs displayed on an exterior of the case.

10. A method for using the apparatus according to claim 1 for sanitization of the wearable equipment used in the catalyst reactor field.

11. A method for disinfecting a helmet comprising the steps of:
vibrating an amount of liquid disinfectant via an atomizer;
creating a fog of disinfectant as a result of the vibrating; and
propelling the fog of disinfectant to the helmet.

12. The method of claim 11, further comprising the steps of containing the atomizer and the helmet in a case having a first chamber and a second chamber; wherein the atomizer is in the first chamber of the case, and wherein the helmet is in the second chamber of the case.

13. The method of claim 12, wherein the atomizer is housed within an internal assembly housing, and further comprising the steps of providing a condenser within the internal assembly housing and an electronics control within the internal assembly housing; and the fog is propelled to the helmet via a port on the internal assembly housing.

14. The method of claim 13, further comprising the steps of collecting a volume of humid air and excess fog from the second chamber to the condenser.

15. The method of claim 14, further comprising the steps of removing condensation from the volume of humid air via the condenser and providing a volume of drier air to the atomizer.

16. The method of claim 15, further comprising the step of providing an air flow to move the fog of disinfectant, the volume of humid air, and the volume of drier air via a fan located within the condenser.

17. The method of claim 16, wherein the amount of liquid disinfectant and the fog of disinfectant is 8% hydrogen peroxide.

18. The method of claim 17, further comprising the steps of
providing a tubing having an exhalation valve plug on a first end and a second end;
inserting the exhalation valve plug into an exhalation valve opening on the helmet;
inserting the second end into a sensing port of the helmet.

19. The method of claim 18, further comprising the step of drawing the air flow from the second chamber via a circulating pump located in the internal assembly housing, wherein the plugged exhalation valve opening of the helmet and the sensing port as connected via the tubing provides the necessary suction to draw the fog of disinfectant through an internal cavity of the helmet.

20. The method according to claim 19, further comprising the steps of successfully sanitizing the helmet from communicable diseases via the fog of disinfectant; and
removing the fog of disinfectant from the second chamber.

21. The method according to claim 20, wherein the following earlier steps are completed within 5 to 10 minutes: vibrating the amount of liquid disinfectant via the atomizer; creating the fog of disinfectant as a result of the vibrating; propelling the fog of disinfectant to the helmet; successfully sanitizing the helmet from communicable diseases via the fog of disinfectant; and removing the fog of disinfectant from the second chamber.

22. An apparatus for disinfecting a helmet in the catalyst reactor field comprising:
a case having a first chamber and a second chamber, wherein the helmet is located in the second chamber;
an internal assembly located within the first chamber of the case;
a condenser within the internal assembly, wherein the condenser is configured to dehumidify an airflow between the condenser and the second chamber;
an atomizer assembly within the internal assembly and connected to the condenser, wherein the atomizer assembly comprises an atomizer chamber configured to be filled with a liquid disinfectant, and wherein the atomizer assembly is configured to convert the liquid disinfectant into a fog of disinfectant;
a circulating pump within the internal assembly, wherein the circulating pump is configured to draw the airflow from an inside of the helmet;
an electronics controls configured to communicate with and time the condenser, atomizer, and circulating pump;
wherein the internal assembly comprises a housing containing the condenser, the atomizer assembly, the circulating pump, and the electronics controls, and wherein a disinfectant port is defined on a side of the housing adjacent to the second chamber of the case, and further comprising at least one vibration damper securing the housing to the case; and
wherein the liquid disinfectant is 8% hydrogen peroxide.

23. A sanitation apparatus for disinfecting a wearable equipment used in the catalyst reactor field comprising:
a case having a first chamber and a second chamber;
an internal assembly located within the first chamber of the case;
an atomizer assembly within the internal assembly;
a fan within the internal assembly;
a programmable logic controller configured to communicate with the atomizer, and the fan; and
wherein the internal assembly comprises a housing containing the atomizer assembly, the fan, and a pool of a liquid disinfectant accumulated at a floor of the housing;
a disinfectant port is defined through the housing, adjacent to, and into the second chamber of the case; and
wherein the second chamber is configured for mounting of the wearable equipment.
